(19) United States
(12) Patent Application Publication
(10) Pub. No.: US 2019/0029654 A1
(43) Pub. Date: Jan. 31, 2019

(54) ADVANCED ELECTROPORATION DEVICES AND METHODS FOR ANALYTE ACCESS IN BIOFLUIDS

(71) Applicants: University Of Cincinnati, Cincinnati, OH (US); Eccrine Systems, Inc., Cincinnati, OH (US)

(72) Inventors: Jason C. Heikenfeld, Cincinnati, OH (US); Richard Heller, Cincinnati, OH (US)

(73) Assignees: University Of Cincinnati, Cincinnati, OH (US); Eccrine Systems, Inc., Cincinnati, OH (US)

(21) Appl. No.: 16/069,658

(22) PCT Filed: Jan. 13, 2017
(86) PCT No.: PCT/US17/13453

§ 371 Date: Jul. 12, 2018

Related U.S. Application Data
(60) Provisional application No. 62/279,189, filed on Jan. 15, 2016.
(60) Provisional application No. 62/307,131, filed on Mar. 11, 2016.

(51) Int. Cl. A61B 10/00; A61B 5/145; A61B 5/00; A61B 5/053; A61B 3/10; A61B 5/1477

(52) U.S. Cl. CPC A61B 10/0064; A61B 5/14546; A61B 5/4266; A61B 5/0531; A61B 2010/0067; A61B 3/101; A61B 10/0051; A61B 5/1477; A61B 5/14521; A61B 5/4277

(57) ABSTRACT

A method of collecting and sensing a biofluid with enhanced concentration of analytes due to electroporation comprises electroporating biofluid glands (14) that are generating a biofluid and specifically sensing at least one analyte in said biofluid, the at least one analyte having a molecular weight greater than 50 Da. A device (100) wearable on a user's skin (12) for receiving an advective flow of a biofluid comprises at least one of a biofluid stimulation component (140), a biofluid sensor (220, 222) specific to an analyte, or a biofluid collection element (230, 232), at least one electroporation electrode (290) for enhancing concentration of at least one analyte in the biofluid having a molecular weight of greater than 50 Da, a counter electrode (195), and an electroporation waveform generator configured to cause the electroporation electrode (290) to generate and direct a plurality of electroporation pulses into the skin (12).

### CROSS-REFERENCE TO RELATED APPLICATIONS

This application is the U.S. National Phase Application claiming priority to PCT Application No. PCT/US2017/013453 filed Jan. 13, 2017 which claims priority to U.S. Provisional Application Nos. 62/279,189 filed Jan. 15, 2016 and 62/307,131 filed Mar. 11, 2016, the disclosures of which are hereby incorporated by reference herein in their entirety.

### BACKGROUND OF THE INVENTION

Sweat and saliva sensing technologies have enormous potential for applications ranging from athletics, to neonatology, to pharmacological monitoring, to personal digital health, to name a few applications. Sweat and saliva contain many of the same biomarkers, chemicals, or solutes that are carried in blood and can provide significant information enabling one to diagnose ailments, health status, toxins, performance, and other physiological attributes even in advance of any physical sign. Furthermore, sweat or saliva itself, the action of sweating or salivating, and other parameters, attributes, solutes, or features on, near, or beneath the skin or oral mucosa can be measured to further reveal physiological information. Moreover, sensing sweat and saliva is relatively non-invasive compared to other biofluids.

However, some solutes such as molecules (e.g., 100's to 1000's Da) and proteins (e.g., 10,000's Da) are dilute in sweat and saliva compared to their concentrations in plasma, often due to the effects of the larger size or a lack of lipophilicity on filtration. Accessing and analyzing such large molecules in sweat or saliva has proven to be difficult. Such analytes can be accessed through microneedles for analysis, but microneedles are invasive. Such analytes can also be accessed through electroporation of the stratum corneum, but electroporation of the stratum corneum can cause a pathway for infection and can include pain or discomfort. Therefore, improved methods of sophisticated and effective integration and application of sweat stimulation, sweat or saliva collection, and sweat or saliva sensing are needed to address one or more of these drawbacks.

### SUMMARY OF THE INVENTION

Embodiments of the disclosed invention provide a sweat or saliva sensor device capable of high performance sweat stimulation, electroporation, and/or sweat or saliva sensing at the same site. Elements of the disclosed invention may be used in combination or in some cases individually.

In an embodiment, a method of collecting and sensing a biofluid with enhanced concentration of analytes due to electroporation is provided. The method comprises electroporating biofluid glands that are generating a biofluid and specifically sensing at least one analyte in said biofluid, the at least one analyte having a molecular weight greater than 50 Da.

In another embodiment, a method of collecting and sensing saliva with enhanced concentration of analytes due to electroporation is provided. The method comprises electroporating saliva glands and specifically sensing at least one analyte in said saliva.

In another embodiment, a device wearable on a user's skin for receiving an advective flow of a biofluid, wherein the biofluid is one of sweat, saliva, and tears, is provided. The device comprises at least one of a biofluid stimulation component, a biofluid sensor specific to an analyte, or a biofluid collection element, at least one electroporation electrode for enhancing concentration of at least one analyte in the biofluid having a molecular weight of greater than 50 Da, a counter electrode, and an electroporation waveform generator configured to cause the electroporation electrode to generate and direct a plurality of electroporation pulses into the skin.

### BRIEF DESCRIPTION OF THE DRAWINGS

The objects and advantages of the disclosed invention will be further appreciated in light of the following detailed descriptions and drawings in which:

FIG. 1 is a top view of a device for sensing a biofluid according to an embodiment.

FIG. 2A is a cross-sectional view of the device taken along the line 2A-2A in FIG. 1 showing a sensing component in contact with the skin.

FIG. 2B is a cross-sectional view of the device in FIG. 2A showing a stimulating component in contact with the skin.

FIG. 3 is a cross-sectional view of the device taken along the line 3-3 in FIG. 1 showing the stimulating component in contact with the skin.

FIG. 4 is a cross-sectional view of a sensing component of the device of FIG. 1 according to an embodiment including elements for electroporation, sweat collection, and sweat sensing.

FIG. 5A is a cross-sectional side-view of a sensing component of the device of FIG. 1 according to an embodiment including elements for electroporation, sweat collection, and sweat sensing.

FIG. 5B is a cross-sectional top-view of a portion of the sensing component of FIG. 5A showing the electrode elements in contact with the skin.

FIG. 6A is a schematic side-view of an electric field surrounding sweat ducts near the skin surface.

FIG. 6B is a schematic top-view of the electric field of FIG. 6A.

FIG. 6C is a schematic side-view of interdigitated electrodes for electroporation according to an embodiment.

FIG. 7 is cross-sectional view of a device for sensing a biofluid according to an embodiment including elements for electroporation and sweat sensing.

FIG. 8 is a cross-sectional view of a device for sensing a biofluid according to an embodiment including elements for electroporation and saliva sensing.

FIG. 9 is a cross-sectional view of a device for sensing a biofluid according to an embodiment including elements for electroporation and saliva collection.

### DEFINITIONS

The definitions below are provided in the context of sweat and sweat glands, but also apply to saliva, salivary glands, tears, and tear ducts in the context of the disclosed invention.

As used herein, “chronological assurance” means using a sweat sensor device to measure a sweat analyte so that the measurement reflects the analyte's concentration in a fresh sweat sample as it emerges from skin. By contrast, a sweat analyte measurement lacking chronological assurance may reflect the analyte's concentration in a sweat sample consisting of fresh sweat mixed with older sweat. Determining chronological assurance may consider how a particular measurement is affected by potential contamination with previously generated sweat, previously generated solutes, other fluid, or other contamination sources.

As used herein, “continuous monitoring” means the capability of a device to provide at least one measurement of sweat determined by a continuous or multiple collection and sensing of that measurement or to provide a plurality of measurements of sweat over time.

As used herein, “determined” may encompass more specific meanings including but not limited to: something that is predetermined before use of a device; something that is determined during use of a device; and something that could be a combination of determinations made before and during use of a device.

As used herein, “sweat sampling rate” is the effective rate at which new sweat or sweat solutes, originating from the sweat gland or from skin or tissue, reaches a sensor that measures a property of sweat or its solutes. Sweat sampling rate, in some cases, can be far more complex than just sweat generation rate. Sweat sampling rate directly determines or is a contributing factor in determining the chronological assurance. Times and rates are inversely proportional (rates having at least partial units of 1/seconds), therefore a short or small time required to refill a sweat volume can also be said to have a fast or high sweat sampling rate. The inverse of sweat sampling rate (1/s) could also be interpreted as a “sweat sampling interval” (s). Sweat sampling rates or intervals are not necessarily regular, discrete, periodic, discontinuous, or subject to other limitations. Like chronological assurance, sweat sampling rate may also include a determination of the effect of potential contamination with previously generated sweat, previously generated solutes, other fluid, or other measurement contamination sources for the measurement(s). Sweat sampling rate can also be in whole or in part determined from solute generation, transport, advective transport of fluid, diffusion transport of solutes, or other factors that will impact the rate at which new sweat or sweat solutes reach a sensor and/or are altered by older sweat or solutes or other contamination sources. Sensor response times may also affect sampling rate.

As used herein, “sweat stimulation” is the direct or indirect causing of sweat generation by any external stimulus such as chemical, heat, optical, electrical current, or other methods, with the external stimulus being applied for the purpose of stimulating sweat. One example of sweat stimulation is the administration of a sweat stimulant such as pilocarpine, acetylcholine, methacholine, carbachol, bethanechol, or other suitable chemical stimulant by iontophoresis, diffusion, injection, ingestion, or other suitable means. Some sweat stimulants are effective for a period of minutes, hours, or more. Generally, longer lasting sweat stimulation methods minimize mechanical re-arrangement of components during use. Sweat stimulation may also include sudo-motor axon reflex sweating, where the stimulation site and sweat generation site are not the same but in close in proximity and physiologically linked in the sweat response.

As used herein, a “sweat stimulating component” or “sweat stimulation component” is any component or material that is capable of locally stimulating sweat to a rate greater than the natural local rate if such stimulation were not applied locally to the body.

As used herein, a “sweat sensing component” or “sweat sensor component” is any component or material that is capable of sensing sweat, a solute in sweat, a property of sweat, a property of skin due to sweat, or any other thing to be sensed that is in relation to sweat or causes of sweat. Sweat sensing components can include, for example one or multiple sensors such as, potentiometric, amperometric, impedance, optical, mechanical, or other types known by those skilled in the art. A sweat sensing component may also include supporting materials or features for additional purposes, with non-limiting examples including local-buffering of sensor electronic signals or additional components for sweat management such as microfluidic materials.

As used herein, “sweat generation rate” is the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

As used herein, “measured” can imply an exact or precise quantitative measurement and can include broader meanings such as, for example, measuring a relative amount of change of something. Measured can also imply a binary measurement, such as ‘yes’ or ‘no’ type measurements.

As used herein, “sweat sampling events” represents the number of sweat samples per a given unit of time that are able to be measured and produce a physiologically meaningful measurement of sweat. These events could be for a continuous flow of sweat and equivalent to sweat sampling rate. These events could be for a discontinuous flow of sweat, for example the number of times the sweat volume or sweat generation rate are adequate to make a proper sweat measurement. For example, if a person needed to measure cortisol three times per day, then the sweat flow rate would need to be adequate to provide a useful sweat cortisol measurement at least three times in the day, and other times during the day could be greater or lower than that adequate sweat flow rate.

As used herein, “sweat volume” is the fluidic volume in a space that can be defined multiple ways. Sweat volume may be the volume that exists between a sensor and the point of generation of sweat. Sweat volume can include the volume that can be occupied by sweat between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

As used herein, “solute generation rate” is simply the rate at which solutes move from the body or other sources into sweat. “Solute sampling rate” includes the rate at which these solutes reach one or more sensors.

As used herein, “microfluidic components” are channels in polymer, textiles, paper, or other components known in the art for guiding movement of a fluid or at least partial containment of a fluid.

As used herein, “state void of sweat” is where a space or material or surface that can be wetted, filled, or partially filled by sweat is in a state where it is entirely or substantially (e.g., greater than 50%) dry or void of sweat.

As used herein, “advective transport” is a transport mechanism of a substance or conserved property by a fluid due to the fluid's bulk motion.

As used herein, “diffusion” is the net movement of a substance from a region of high concentration to a region of low concentration. This is also referred to as the movement of a substance down a concentration gradient.

As used herein, “convection” is the concerted, collective movement of groups or aggregates of molecules within fluids and rheids, either through advection or through diffusion or a combination of both.

As used herein, a “volume-reduced pathway” is a sweat volume that has been reduced by the addition of a material, device, layer, or other body-foreign substance, which therefore increases the sweat sampling interval for a given sweat generation rate. This term can also be used interchangeably in some cases with a “reduced sweat pathway”, which is a pathway between sweat glands and sensors that is reduced in terms of volume or in terms of surfaces wetted by sweat along the pathway. Volume reduced pathways or reduced sweat pathways include those created by sealing the surface of skin, because skin can absorb or exchange water and solutes in sweat, which could increase the sweat sampling interval and/or cause contamination, which can also alter the accuracy or duration of the sweat sampling interval.

As used herein, “volume reducing component” means any component that reduces the sweat volume. In some cases, the volume reducing component is more than just a volume reducing material, because a volume reducing material by itself may not allow proper device function (e.g., the volume reducing material would need to be isolated from a sensor for which the volume reducing material could damage or degrade, and therefore the volume reducing component may comprise the volume reducing material and at least one additional material or layer to isolate volume reducing material from said sensors).

As used herein, “mechanical co-location” refers to one or more components that can be mechanically moved or arranged in a manner that causes the components to be coupled or de-coupled to a common area of skin (i.e., one or both components are movable relative to the common area of skin), and such that the two or more components during at least one point are carried simultaneously by the device, and such that at least one component is continuously carried by the device during its use. The term “mechanical movement” includes manual movement of device components. For example, a device that places a stimulating component onto skin, removes the stimulating component from skin, and then with a separate device places a sensing component onto skin, does not meet the definition of “mechanical co-location” because neither of these components is always carried by the device, as will be further described in the disclosed invention. For a first example, the definition of “mechanical co-location” would be met by a device that carries a sweat sensing component during use of the device and integrates an iontophoretic sweat stimulating component temporarily, with the stimulating component during stimulation being coupled to at least a common portion of skin to which the sensing component is coupled. For a second example, the definition of “mechanical co-location” would be met by a device that carries an electroporation component and a sensing component during use of the device.

As used herein, “electroporation” refers to electroporation dominantly of the eccrine or apocrine sweat ducts and glands, or other biofluid glands, and excludes electroporation that is dominantly of the stratum corneum. Multiple electroporated pores or pathways are possible, for example pathways through cellular membranes, paracellular pathways (including through tight junctions), or other possible pathways. In one aspect, at least ⅔ of the increase in analyte concentration in sweat may be due to electroporation of the sweat ducts and glands (i.e., the additional analytes do not originate from the skin surface). In another aspect, at least 9/10 of the increase in analyte concentration may be due to electroporation of the sweat ducts and glands (i.e., not from the skin surface). Such values could be validated by testing electroporation with or without sweating, and then testing solute concentrations both with and without active sweating, or estimated by using skin impedance with or without sweating. Such values also could be validated by measuring analytes in sweat with and without one or more methods for reduced sweat volume that can isolate sweat ducts from the skin surface. Electroporation as used herein is dependent on a flow of sweat, and/or a sweat volume filled with sweat to enable a pathway for solutes to reach a sensor. Electroporation therefore excludes electro-osmosis in the absence of sweating, for example, as previously shown for the Glucowatch Biographer product for transdermal glucose monitoring, which is entirely reliant on a continuously applied DC voltage with a current density of about 0.3 mA/cm² to create both a pathway of fluid and a flux of analyte. The GlucoWatch requires a 2 hour warm-up period to reach a steady state flux of glucose by electro-osmosis. Embodiments described below may allow for to analyte signals that are measured in just a few minutes. Electroporation may use any magnitude, frequency, waveform, polarity, current limit, voltage limit, or other voltage or current features or requirements that satisfy the above definition of electroporation. Electroporation may generally include any phenomena that electrically enhances flux into sweat of analytes that are greater than 50 Da in molar mass, and may include physical pores or pathways created in tissue or cell membranes, or between cells, or may include a destabilization of tissues or membranes that allows increased analyte flux. In some cases, electroporation may also increase the flux of analytes, such as ions, that are less than 50 Da. Thus, when electroporation is applied to increase the flux of analytes that are greater than 50 Da, an additional effect may be the increase in flux of analytes that are less than 50 Da.

As used herein, “electroporation waveform” is any waveform for increasing skin permeability that operates by creating physical pores in, or the destabilization of, skin, tissue, or cellular structures thereby increasing flux into sweat of analytes that are greater than 50 Da in molar mass. This increased skin permeability can last for several minutes. Electroporation may be caused entirely or partially by one or more electroporation waveforms. Often, electroporation waveforms are on the order of, but not limited to, 100's to 1000's V/cm. The actual waveform used may vary depending on the specific application, electrode distances, etc. Electroporation waveforms can be monopolar or bipolar (i.e., negative and/or positive).

As used herein, “chaser waveform” is a waveform that may be applied before, during (by waveform superposition), or after an electroporation waveform or between a plurality of electroporation waveforms. Electroporation therefore may be caused partially by one or more chaser waveforms. Chaser waveforms have the purpose of enhancing flux of analytes into sweat that are greater than 50 Da in molar mass through the physical pores caused by an electroporation waveform. Chaser waveforms often have, but are not necessarily limited to, voltages on tissues or cells that are lower than those used for electroporation waveforms. Chaser waveforms may be on the order of, but not limited to, 10's to 100's V/cm. The actual waveform used may vary depending on the specific application, electrode distances, etc. Chaser waveforms can be monopolar or bipolar.

### DETAILED DESCRIPTION OF THE INVENTION

Embodiments of the disclosed invention are directed to biofluid sensing devices capable of high performance biofluid sensing whereby greater solute access is enabled by electroporation. Although various embodiments described below are described as being specific to sweat or saliva, such description may apply to sweat, saliva, and tears even if not explicitly mentioned. Although the discussion for sweat focuses on eccrine sweat glands, it should be recognized that the description may apply to other biofluid gland types, such as apocrine glands, salivary glands, or tear glands. Large analytes may be extracted in sweat or saliva with greater concentrations where electroporation is performed in a manner that is more targeted, precise, and limited only to live tissue lining the eccrine sweat glands or salivary glands in the mouth. For tears, electroporation electrodes or sensors could be provided anywhere on or near the surface of the cornea, the canaliculi, the lacrimal sac, or the lacrimal duct. Such an approach is safe and effectively non-invasive because an outward flow of sweat or saliva prevents infection, and live cells can repair any damage or regenerate quickly (e.g., a minute or less) if the electroporation is adequately controlled. When electroporation can be made more selective to just the live cells lining the sweat ducts or salivary ducts/glands, safer and more repeatable/reliable biomarker extraction and sensing mechanisms can be enabled. Furthermore, specific to sweat, repeatability, reliability, and safety can all be improved if the sweat glands are actively sweating (e.g., filled with salty sweat, which is electrically conductive).

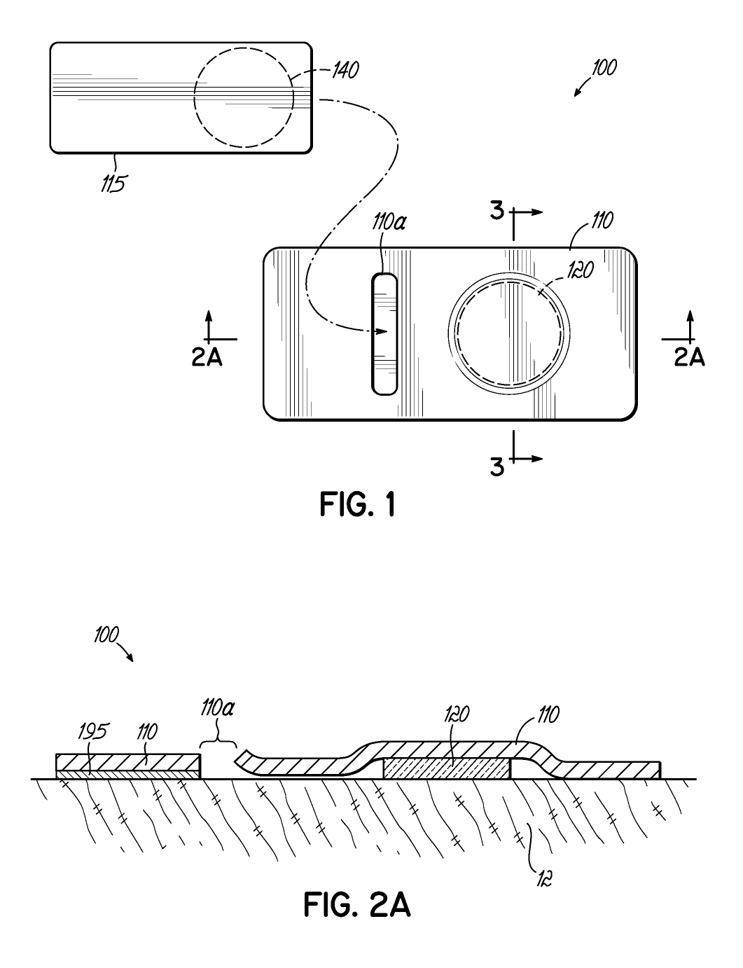

With reference to FIG. 1, in an embodiment of the disclosed invention, a sweat sensing device 100 is shown that is capable of electroporating sweat glands. The device 100 includes: a first substrate 110 including an aperture 110a, a sensing component 120 on the first substrate 110, a second substrate 115, and a stimulation and/or electroporation component 140. The first substrate 110 may be, for example, a flexible plastic film such at PET or textile. The first substrate 110 may also include a medical adhesive to adhere the first substrate 110 to the skin 12. The sensing component 120 may be, for example, an electrical impedance aptamer or antibody sensor for albumin or for luteinizing hormone for example. The second substrate 115 and the stimulation and/or electroporation component 140 are sized to be moved through the aperture 110a. For example, the width of the second substrate 115, or the portion of the second substrate 115 connected to the component 140, may be less than the width of the aperture 110a. The second substrate 115 may be, for example, a plastic film that is semi-rigid. The component 140 is configured to stimulate sweat or other biofluid and can be, for example, an iontophoresis electrode carrying a semi-rigid aragose gel with a chemical sweat stimulant. Stimulation component 140 could also be utilized for electroporation. For example, after iontophoresis is completed for sweat stimulation, a series of short electrical pulses could be applied to cause electroporation. The sensing component 120, which is configured to sense sweat and electroporate the skin 12, is described in greater detail below, and may include electronics, sweat sensors, microfluidics, or other suitable or improving elements, designs, or features.

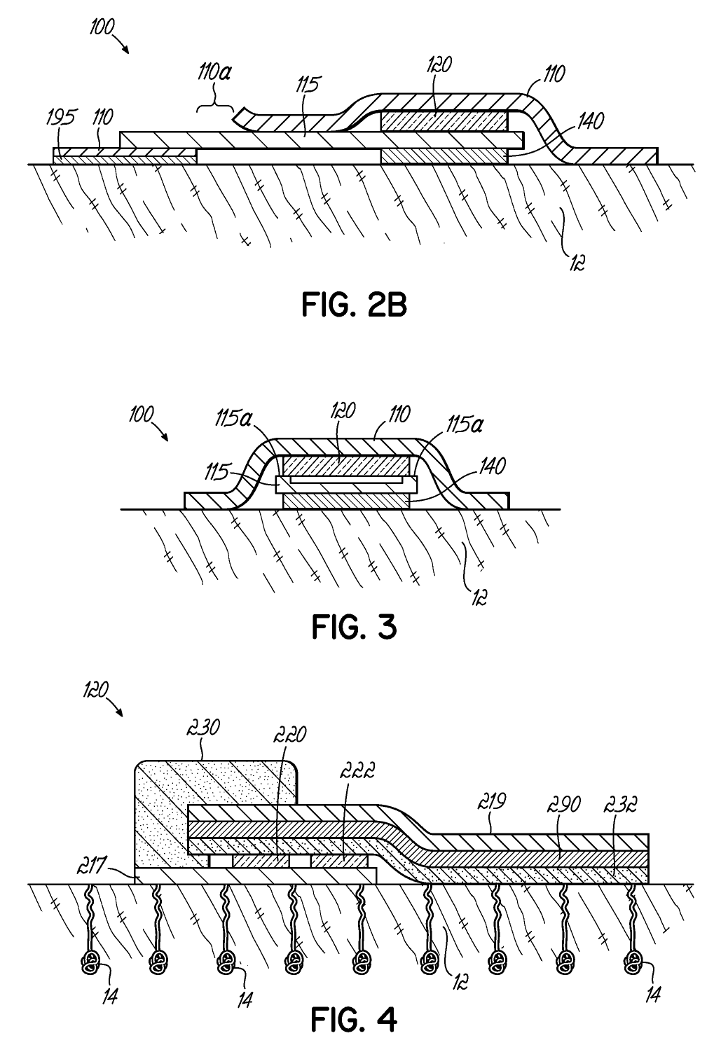

With reference to FIG. 2A, the device 100 is shown prior to the insertion of the second substrate 115. As shown, the sensing component 120 is near to, or intimate with, the skin 12. Additionally, a counter electrode 195 is positioned between the first substrate 110 and the skin 12. The counter electrode 195 can be, for example, a counter electrode for electroporation or iontophoresis.

With reference to FIGS. 2B and 3, the device 100 is shown after the second substrate 115 and the stimulation component 140 have been mechanically moved through the aperture 110a to an activated position on the skin 12 previously occupied by the sensing component 120. In other words, when the second substrate 115 is in the activated position, the stimulation component 140 occupies the area of the skin 12 that was previously occupied by the sensing component 120. In this configuration, the stimulation component 140 can then stimulate sweat by, for example, iontophoresis of a sweat stimulant, or apply electroporation, as previously described. Next, the second substrate 115 and stimulation component 140 are removed from the aperture 110a to return the device 100 to the configuration of FIG. 2A where the sensing component 120 is once again in contact with the skin 12, and senses the stimulated sweat. This process can be repeated based on need for sweat, based on need for a measurement, or as determined by any method or schedule. For example, in an embodiment where the stimulation component 140 includes the stimulant carbachol, which can induce sweating for 10 hours, stimulation could be applied for two minutes (e.g., using the configuration shown in FIGS. 2B and 3) while the sensing component 120 measures sweat for 9 hours and 58 minutes (e.g., using the configuration shown in FIG. 2A). Mechanical movement of the second substrate 115 can be achieved by the user (e.g., using fingers or a specially designed applicator) or by mechanical motors, tracks, or other mechanical techniques that could be integrated onto the device 100. In another embodiment, the stimulation component 140 and the second substrate 115 could be integrated into the device 100 and moved into and out of place as needed.

The first substrate 110 is configured to (1) hold the sensing component 120 against the skin 12 when the second substrate 115 is not inserted (FIG. 2A), and (2) hold the stimulation component 140 against the skin 12 when the second substrate 115 is inserted (FIGS. 2B and 3). For example, the first substrate may be stretchy or flexible or may stretch the skin 12. Alternatively, or additionally, the first substrate 110 may include, for example, springs, sponges, or other suitable materials to provide pressure to secure one or more components against the skin 12.

With further reference to FIG. 3, the mechanical co-location of components of the device 100 may include materials, features, or methods that protect the sensing component 120 and/or the stimulation component 140 from significant damage during mechanical movement of one or more of such components. More particularly, in an embodiment, the second substrate 115 includes raised portions 115a. The raised portions 115a prevent the second substrate 115 from scraping against the sensing surface of the sensing component 120 when the second substrate 115 is inserted and removed from the aperture 110a. It should be recognized that other techniques may be used to protect the components during the movement of the second substrate 115. For example, textiles or microfluidics may be placed between the sensing component 120 and the skin 12 or the second substrate 115 (not shown). It should be recognized that the configuration of devices according to the disclosed invention may vary.

With reference to FIG. 4, a configuration of the sensing component 120 according to an embodiment is illustrated in greater detail. A substrate 219 carries an electrode 290. The electrode 290 is configured to deliver one or more electroporation voltages or waveforms to the skin 12 and, therefore, to sweat glands 14 in the skin 12. It should be recognized that, in an embodiment where the sensing component 120 includes an electroporation component such as the electroporation electrode 290, the component 140 may optionally be used only to stimulate sweating. For electroporation uses by the electrode 290, a counter-electrode in contact with the body is also required. Therefore, in such embodiments, a counter-electrode could be the electrode 195 depicted in FIG. 2A. Such a counter electrode may have a larger contact area with the skin 12 than the contact area of the electrode 290 with the skin 12, such that electrical stress on the skin 12 is primarily limited to the contact area between the electrode 290 and the skin 12. The electrode 290 may optionally be configured to perform a sensing function. In an embodiment, the electrode 290 could measure skin impedance, which is used to determine sweat rate, the degree of electroporation, or skin healing (i.e., recovery) from electroporation, or the reversing of electroporation. Materials for the electrode 290 include, without limitation, metal, conductive polymer, carbon, or other suitable materials. The electrode 290 could include a pH balancing composition or pH buffering materials (not shown) to prevent significant pH changes during operation (e.g., due to electrolysis of water at the electrode).

With further reference to FIG. 4, a substrate 217 carries sensors 220, 222. The substrate 217 may be, for example, polyester. The sensors 220, 222 can include any type of sweat sensor for sensing analytes such as ions, molecules, or proteins. Exemplary sensor types include ion-selective, amperometric, colorimetric, aptamer, antibody, electrical, optical, mechanical, or other suitable types. The sensors 220, 222 may be analyte-specific and may both sense the same analyte or may sense different analytes. In an embodiment, a sensing component includes at least one analyte-specific sensor for measuring at least one analyte in sweat that is enhanced in concentration in sweat due to electroporation. Generally, but not so limited, analytes that can be increased in concentration in sweat by electroporation will have a molecular weight greater than 50 Da (e.g., excluding electrolytes such as K+, Na+, and Cl−, which all have molecular weights less than 50 Da). In some cases, divalent analytes, although small, may also benefit from electroporation. Even analytes as small as glycerol (92 Da) are diluted in saliva and, at high sweat generation rates (e.g., 1's of nL/min/gland), may be diluted in sweat as well.

Still referring to FIG. 4, the substrate 219 also carries a sweat wicking collector 232. The wicking collector 232 collects sweat that is generated from glands in the skin 12 and transports the sweat to the sensors 220, 222. Although the wicking collector 232 is shown between the skin 12 and the electrode 290, it should be recognized that the locations of the electrode 290 and the wicking collector 232 can be reversed or re-arranged. Further, the electrode 290 and the wicking collector 232 could be combined (not shown). Materials for the wicking collector 232 may include, without limitation, paper, textile, cellulose, beads, gels, microfluidics, or other suitable materials. A wicking pump 230 is also provided to receive sweat from the wicking collector 232 such that a continual flow of sweat is provided across the sensors 220, 222 while the sweat is generated and collected from the skin 12. The wicking pump 230 may be made of similar or different material than the wicking collector 232.

In one embodiment, the electrode 290 is electrically grounded (e.g., using the same electrical ground used by the sensors 220, 222 (not shown)), and the voltage for electroporation is applied by a larger counter electrode (e.g., the electrode 195) located elsewhere on the skin 12. In other words, the electrode is referenced to the same voltage potential as said at least one biofluid sensor. As a result, the sensors 220, 222 do not experience the electroporation voltage that could interfere with or damage the sensors 220, 222. Additionally or alternatively, electronics in the device 100 (not shown) could also use lock-in-amplifying, shielding, or filtering methods to eliminate electrical noise imposed on the sensors 220, 222 by the electroporation voltages.

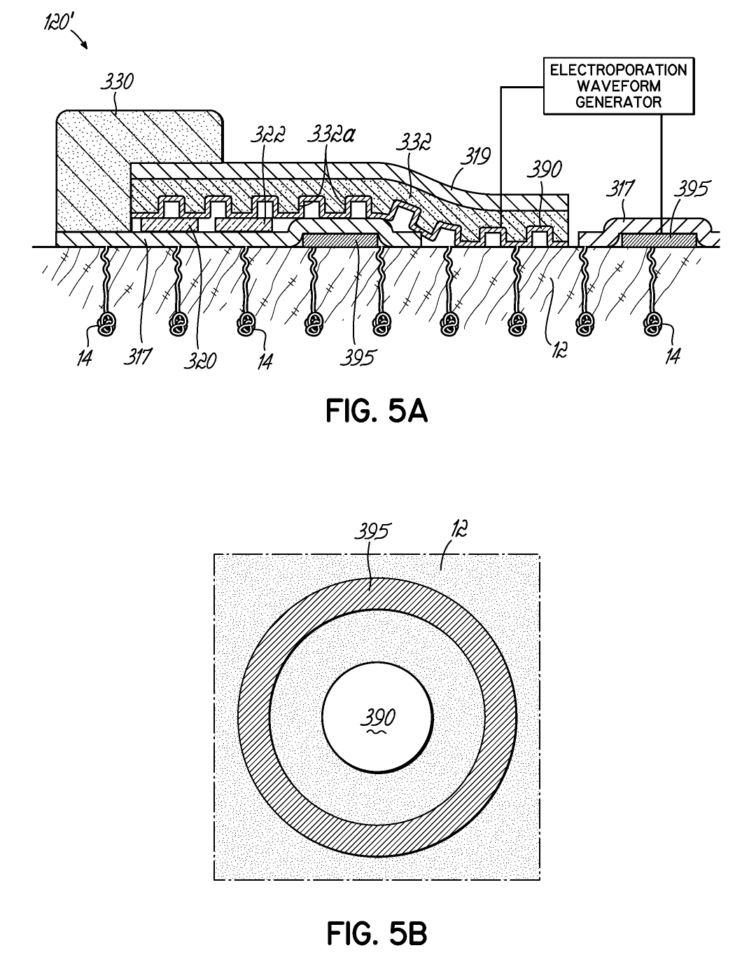

With reference to FIGS. 5A and 5B, where like numerals and features correspond to numerals and features of previous figures, an exemplary configuration of the sensing component 120 is shown. The sensing component 120 includes a substrate 317 that carries sensors 320, 322 and a substrate 319 that carries a wicking component 332 and an electrode 390. The wicking component 332 has a network (e.g., a hexagonal network) of channels 332a that are coated with the electroporation electrode 390. In an embodiment, the wicking component 332 is a microreplicated polymer, and the electroporation electrode 390 is made of hydrophilic gold. The electrode 390 can also be textured to achieve greater hydrophilicity, be coated with a hydrogel, such as agar, or be coated with a hydrophilic monolayer, for example. These channels 332a form a wicking component for reduced sweat volume. This particular configuration has an advantage of reducing the sweat volume, as taught in U.S. Provisional Application No. 62/196,541, the disclosure of which is incorporated by reference herein in its entirety. The counter electrode 395 is closer to the electroporation electrode 390 as compared to the electrodes 290, 195 (shown in FIGS. 2B and 4), which could reduce the required electroporation voltage relative to those configurations. FIG. 5B provides a top-view of the electrodes 395 and 390 as they are in contact with skin (the portions not in contact with skin are not shown). Having a counter electrode 395 that surrounds the electroporation electrode 390 advantageously provides a uniform electric field to more of the sweat glands 14 covered by the electroporation electrode 390.

With further reference to FIG. 5A, in an aspect of the disclosed invention, the electroporation may be controlled by an electroporation waveform generator. For example, the electroporation electrode 390 and the counter electrode 395 may be coupled to the electroporation waveform generator, which may also be referred to as a controller. The electroporation waveform generator is configured to control the electroporation applied to the skin 12 by causing the electroporation electrode 590 to generate a plurality of electroporation pulses, which are directed into the skin 12. The electroporation waveform generator may be configured to cause the generation of chaser waveforms in addition to electroporation waveforms. It should be recognized that the electroporation waveform generator may be combined with other aspects and features described herein. For example, an electroporation waveform generator may be coupled to the electroporation electrode 290 and the counter electrode 195 of the device 100.

An exemplary method of using the device 100 is now described. First, sweat stimulation is induced as shown in FIGS. 1-2B at the site occupied by the sensing component 120. Alternately, sweat could be naturally generated (e.g., due to exercise, heat, or anxiety). Secondly, the sensing component 120 is placed at the site of skin 12 that was stimulated to create sweat, for example by removing the substrate 115 and the stimulation component 140 from the aperture 110a. At this point, active sweating is occurring, and with further reference to FIG. 4, the generated sweat is transported from the skin 12 towards the sensors 220, 222 by the wicking collector 232. The presence of the collected sweat could be sensed by the electrode 290, by the sensors 220, 222, or by another mechanism. Once sweating is confirmed, the electrode 290 is conductive with the sweat in the wicking collector 232 and, therefore, is conductive with the sweat glands 14. The electrical connection between the electrode 290 and the sweat glands 14 allows electroporation of the sweat glands 14. An electroporation waveform generator (as shown in FIG. 5A) may cause the electrode 290 to generate a plurality of electroporation pulses that are directed into the skin 12. The electroporation may include at least one electroporation waveform and may optionally include one or more chaser waveforms. Once a proper level of electroporation is achieved, analytes can be advectively, convectively, diffusively, iontophoretically, electrophoretically, or electro-osmotically transported to the sensors 220, 222. During sensing of the generated sweat, an advective flow of sweat caused by sweat generation by one or more sweat glands is maintained. In other words, analytes are transferred to the sensors 220, 222 to be sensed at least in part by the bulk flow of sweat.

It should be recognized that the electroporation voltage may vary based on the intended application. In an embodiment, a 1 to 3 V bipolar electroporation voltage could be applied for 1 ms duration at a frequency of repeated pulses or voltage magnitude needed to maintain a certain degree of electroporation. In an embodiment, electroporation is intermittently applied at voltages of 500 mV or greater. If the sweat ducts are targeted for electroporation, a voltage of about 2 to 4 V may be required to penetrate the double cell lining of the sweat ducts. A similar case may exist for salivary glands. At 2 to 4 V, electroporation can enhance partitioning of solutes from the targeted live cells and tissue by one or more orders of magnitude. However, where the stratum corneum of the skin or oral mucosa is the primary target, an electroporation voltage of about 30 to 100 V or more is typically utilized, which is far more damaging to the surrounding cells. In one embodiment, electroporation voltages could be increased to values that allow greater analyte concentrations and/or greater analyte sizes at which the user could begin to sense the presence of the voltage. Therefore, in such cases, a numbing, anti-inflammatory, or pain-relieving agent (such as Oragel) could be topically or iontophoretically applied before or during placement of the devices.

It should be recognized that the characteristics of the applied electroporation voltage may vary based on the intended application. In an aspect of the disclosed invention, the use of various polarities, frequencies, magnitudes, waveforms, and other methods of altering the electroporation voltage application could enhance electroporation specific to certain types or sizes of analytes. Further, electroporation can target different ratios of enhancement of solutes that may come from intra-cellular or from extra-cellular regions of tissue. For example, extracellular glucose may be a preferred analyte to measure compared to intracellular glucose. Different pulse magnitudes and widths can be used to control the depth of electroporation into the sweat gland or other target location (e.g., see Example 4). For example, it may be preferable to only or primarily electroporate the dermal duct so as to not interfere with the secretory portion of the gland. It should be recognized that the voltage applied to the skin is greater than the voltage that reaches the desired location (e.g., a certain length of the dermal duct). In an embodiment, a safety level is set, such as a maximum frequency of repeated pulses or a maximum voltage magnitude, that is not to be exceeded (e.g., see Example 4) in order to avoid damaging the skin or specific skin structures. The electroporation pulses may be of various durations, depending on the application, and may be for example, less than 10 s, less than 1 s, less than 100 ms, less than 10 ms, less than 1 ms, less than 100 μs, less than 10 μs, or less than 1 μs.

Electroporation may be applied once, intermittently (e.g., AC, pulsed DC, etc.), continuously, or as needed by the electrode 290. In an embodiment of the disclosed invention, electroporation may be applied periodically or on demand, rather than continuously, to reduce stress on the body or to reduce interference with the secretory coil, which can inhibit sweat generation. With respect to sweat inhibition, a continued flow of current into the skin can inhibit sweating (e.g., iontophoresis is commonly used to treat hyperhidrosis). This could negatively impact device applications that require sweat stimulation, especially where the electroporation is more aggressive to obtain a higher concentration or larger-size of analytes into sweat. Similar need to reduce stress could exist in cases where a person is particularly sensitive to, or has some physiological problem with, electroporation. Two exemplary embodiments are now provided.

In an aspect of the disclosed invention, one or more types of waveforms may be applied. As previously described, two such waveforms include an electroporation waveform and a chaser waveform. For example, an electroporation waveform could be applied as a bipolar DC square wave (+/−4 V) with a period of 10's of μs and a total of 8 pulses for each polarity, corresponding to a total of 100 to 200 μs of voltage application. Alternately, a similar waveform with a lower duty cycle could be applied using a positive 10 μs long pulse of 4 V at 0 s, and a −4 V and 10 μs pulse at 0.5 s, and repeating 8 times (i.e., 8 seconds total). The resulting permeabilization of the lining of the eccrine duct can typically last for several minutes. It should be recognized that the effective length of the permeabilization may be shorter or longer depending on the waveform applied, the specific target analyte, or other factors.

Merely because the lining of the eccrine duct is permeabilized by an electroporation waveform does not mean that most or all analytes will easily traverse it. Therefore, in an embodiment, a chaser waveform may be applied to increase the flux of analytes through the permeabilized lining of the eccrine duct. The chaser waveform may be applied after or between electroporation waveforms to continue or enhance entry of analytes into the sweat and to increase the concentration of the analytes in the sweat. This enhancement could be due to iontophoretic effects for charged analytes or for non-charged analytes due to localized flow and related drag forces imparted by charged solutes near the non-charged analytes. The chaser waveform could be AC, DC, pulsed, monopolar, bipolar, or any other suitable type of waveform. For example, because large analytes are highly dilute in sweat, if a first polarity for the chaser waveform pulled in the analyte into the lumen of the sweat duct, a second and opposite polarity could be applied later (milliseconds to seconds to minutes), which would not appreciably remove the large analyte from sweat in the lumen because the large analyte has already diffused away from the pore vicinity or has been advectively transported away by the flow of sweat in the lumen.

As disclosed, an electroporation component may deploy a plurality of different electroporation waveforms and chaser waveforms, and may deploy those various electroporation waveforms and chaser waveforms a plurality of times. In various embodiments, therefore, electroporation and chaser waveforms may be bipolar with equal magnitudes for each polarity, asymmetric in magnitude, or may be monopolar and similar or opposite in polarity or magnitude. Further, because sweat pH and/or salinity could alter the efficacy of the electroporation waveform and/or the chaser waveform, these waveforms could be adjusted as needed. For example, the adjustments to the waveforms may be based on one or more measurements taken by device components in communication with an electroporation component, including sensors providing measurements for analyte concentration, skin impedance, sweat pH, sweat salinity, or other measurements.

In an aspect of the disclosed invention, concentration ratios of two or more analytes can be measured and can be compared over time instead of, or in addition to, comparing the absolute concentrations of each analyte over time. In that regard, for some analytes, especially the larger sized analytes or more dilute analytes, electroporation will be unable to provide concentrations of a particular analyte that are similar to the concentration of that analyte in plasma, cells, intracellular fluid, or other fluid of interest. The two or more analytes would be chosen from those that have similar enhancement of concentration in sweat due to electroporation (e.g., due to a similar size, charge, hydrophilicity, shape, etc.). Measuring and comparing the changes in a concentration ratio of a reference analyte and the target analyte allows for determination of a condition, disease, or other factor in the body and, in some cases, is preferable to measuring the concentration of the analyte itself directly in blood. Exemplary applications include measuring concentrations of cortisol and dehydroepiandrosterone sulfate (DHEAS) and determining a cortisol/DHEAS ratio, which is of interest for stress monitoring, or determining ratios of two cytokines (e.g., one pro-inflammatory, one anti-inflammatory). In an embodiment, a sensor could be provided for each of IL-1 beta (e.g., sensor 220) and for TNF-alpha (e.g., sensor 222), which have similar molecular weights of 17 kDa and similar effective diffusivities, and their ratios compared over time. Other examples of cytokine ratios or other analyte ratios are possible.

In an aspect of the disclosed invention, devices can be utilized to collect sweat without onboard sensors for sensing the targeted analyte. To that end, the device may only include those features and elements useful for electroporation and for collecting an appropriate sample of sweat (e.g., a sweat collection element), which can be analyzed by a sensor or technique outside the device. For example, a device of the disclosed invention could include a microfluidic component for collecting sweat, such as the wicking components 230, 232 shown in FIG. 4.

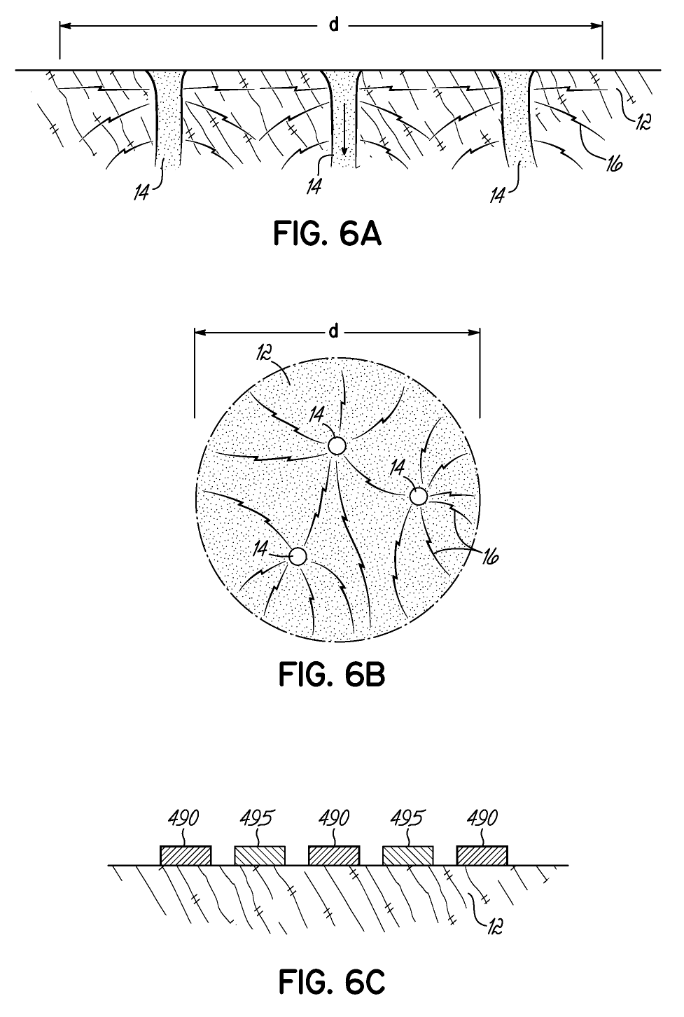

With reference to FIGS. 6A and 6B, diagrams of the dermal ducts of the sweat glands 14 near the surface of the skin 12 and electric field lines 16 during electroporation are provided for reference. As a larger area or a larger number of ducts are electroporated, a greater voltage or duration of electroporation will be needed. In addition, if the electric field has a strong horizontal component (i.e., parallel to the skin surface), then sweat ducts can act as conductors that can partially screen other eccrine ducts from receiving adequate electroporation voltage. Such screening of the electric field can be mitigated using a variety of techniques. For example, a large counter electrode allows deeper vertical penetration of the electric field into the body. Additionally, the location of the counter electrode affects the penetration of the electric field. In an embodiment, a counter electrode may be positioned opposite the skin surface of interest (e.g., electroporation electrode on one side of an arm, and the counter electrode on the other side). For example, a device of the disclosed invention could be held on the arm in contact with the skin by a large, electrically conductive wrap textile that acts as the counter electrode. For another example, one or more counter electrode(s) may be provided on the side(s) of the arm and an electroporation electrode on the center of the arm. In an embodiment, the electroporation element is positioned at least 90 degrees around the body or body part from a skin surface contacted by the counter electrode. Alternately, as shown in FIG. 6C a plurality of electroporation electrodes 490 and counter electrodes 495 can be interspersed (or interdigitated) to localize the electric field. For example, in an embodiment, at 100 glands/cm² with an average gland spacing of 1 mm, alternating electroporation and counter electrodes could be located every 500 μm in distance.

In an aspect of the disclosed invention, electroporation may be applied to more than one location on the skin because electroporation could cause stress on the skin. In an embodiment, a single device (e.g., device 100) could be placed on the skin and moved to a new position on skin so that the device senses sweat and electroporates at more than one location. For example, the elements 219, 290, 232 could be physically moved within a device 100 to alternate locations during use of the device 100. Alternately, a device may include multiple sets of the elements 219, 290, 232 where each set is positioned at a unique location on skin to wick sweat to a common set of sensors 220, 222. In another embodiment, a device could include multiple subcomponents 120, each at a unique location on skin. Applying the electroporation at more than one location on the skin may improve the comfort level for the user.

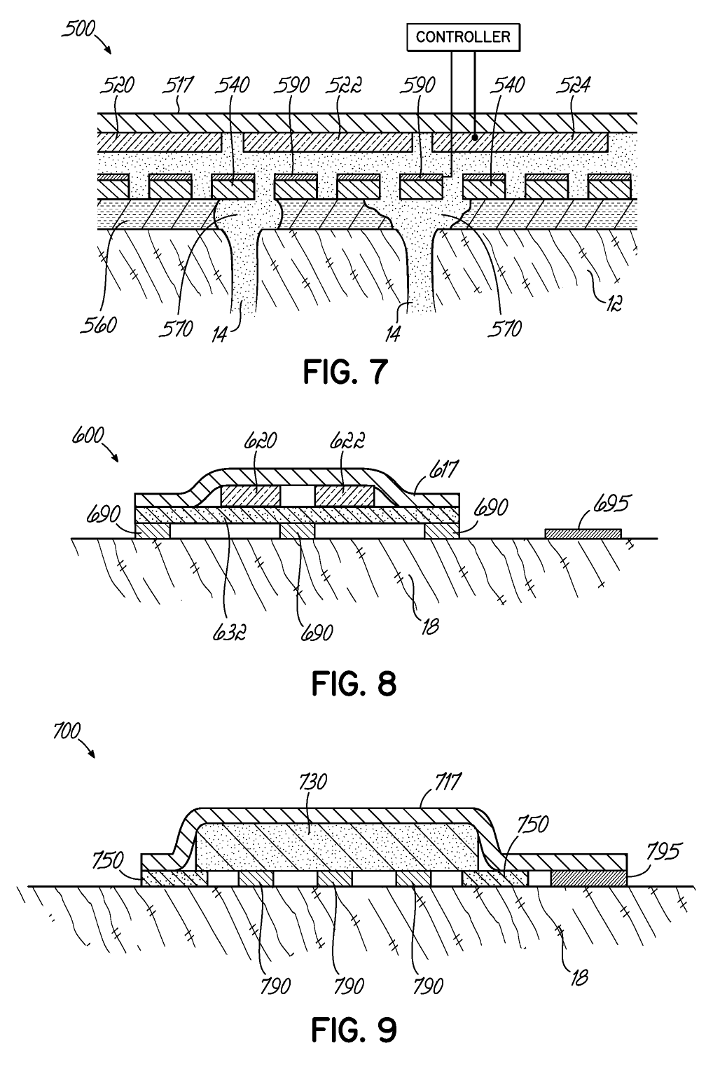

With reference to FIG. 7, in an embodiment, where like numerals and features correspond to numerals and features of previous figures, a device 500 includes sensors 520, 522, 524 carried by a substrate 517 and an electroporation electrode 590. Although not shown, it should be recognized that the device 500 may include a counter electrode and an electroporation waveform generator. The device 500 operates using naturally stimulated sweat. A sweat permeable membrane 540 and a sweat impermeable material 560 are provided to reduce the sweat volume of the device 500 (i.e., the sweat volume between the sensors 520, 522, 524 and the skin 12). The reduced sweat volume is described in more detail in International Patent Application No. PCT/US2015/032893, the disclosure of which is incorporated by reference herein in its entirety. The sweat permeable membrane 540 is coated with the electroporation electrode 590. In an embodiment where the sweat impermeable material 560 may interfere with the sensors 520, 522, 524 (e.g., if the sweat impermeable material 560 is an oil or jelly), the sweat permeable membrane 540 ensures that the sweat impermeable material 560 is kept from the reaching the sensors 520, 522, 524. As illustrated, the sweat 570 creates pathways through the sweat impermeable material 560 and fills the reduced sweat volume, which consequently reduces the area of the skin 12 that is in direct electrical contact with the waveforms provided by the electroporation electrode 590. This could reduce contamination coming from the skin surface, dead skin cells, microbes, or other contamination sources. The reduction in the area of the skin 12 that is in direct electrical contact could also reduce discomfort or other undesired effects caused by any electroporation of the stratum corneum, hair follicles, or defects in the stratum corneum.

In an aspect of the disclosed invention, the electrode sizes and contact area with skin may be designed to mitigate issues with pain or discomfort caused by electrical current passing into the skin 12. Pain or discomfort caused by electrical current in skin does not scale linearly in terms of the relationship of current density to electrode area. The smaller the electrode area that contacts skin is, generally the larger the current density that can be used without a perception of the current or perception of pain. For example, an electrode of 24 cm² area generates a tingle at 0.08 mA/cm², whereas an electrode of 0.64 cm² generates a tingle at 0.4 mA/cm² (varies based on location on skin and from person to person). In an aspect of the disclosed invention, due to the reduced sample volumes, the areas of electrical contact with skin for reverse iontophoresis are reduced, as discussed above. Consider for example, sampling biofluid from pre-existing pathways that are sweat ducts with densities of 100 glands/cm².—The contact areas needed to cover an average of 5, 10, and 50 glands, therefore, would be 0.05 cm², 0.1 cm², and 0.5 cm², respectively. With sweat ducts at densities of 200 glands/cm², then the contact areas needed to cover an average of 5, 10, and 50 glands would be 0.025 cm², 0.05 cm², and 0.25 cm², respectively. Even fewer glands could be covered, so the above areas of contact may represent upper or lower limits for contact areas for one or more embodiments of the disclosed invention. These areas can be of the electrodes themselves or, in the case of intervening materials or layers between the electrodes and skin, can represent the electrical contact area with skin.

With further reference to FIG. 7, in an embodiment, the amount of electroporation is measured and controlled (e.g., using feedback control). Feedback control may involve, without limitation, the measurement of skin impedance by the electrode 590 or the measurement of increased analyte concentrations due to electroporation by one or more of the sensors 520, 522, 524. For example, the electrode 590 and the sensor 524 may be coupled to a controller or computing component, which is configured to control the electroporation applied by the electrode 590 based on measurements by the sensor 524. Feedback control could be used to maintain a reduction in skin impedance that is, for example, no more than 20%, or no more than 50%, or no more than 80% below that of skin impedance without electroporation. In an embodiment, the skin impedance may be measured before electroporation occurs, and may then be compared to skin impedance measured during or after electroporation to determine a reduction in the skin impedance.

In an aspect of the disclosed invention, a device may be configured to apply electroporation only when it would likely result in the desired increase of the target analyte. In that regard, electroporation would be ineffective to increase the flux of the target analyte if there is no presence or flow of sweat. Similarly, some sweat generation rates could be so high (e.g., several nL/min/gland) that analyte concentrations in sweat are too dilute to be sensed, even with application of electroporation. Therefore, a device could measure an advective flow of sweat from the sweat ducts in a binary yes/no format (e.g., is sweat flowing or not) or measure the magnitude of the flow of sweat (e.g., the sweat generation rate in nL/min/gland). Such a yes/no determination could be made by measuring skin impedance, measuring sweat sodium (Na+) concentration, using a microfluidic flow meter or thermal flow meter, or other suitable methods. This additional flow measurement could allow electroporation to be applied by the device only when it would be useful (i.e., in the presence of sweat or when an adequate or non-excessive sweat flow is occurring). To that end, the flow measurement component may be in electronic communication with the electroporation components of the device. In other words, the electroporation components may be controlled using feedback from the flow measurement component. For example, with reference again to FIG. 7, in an embodiment, the device 500 could measure sweat generation rate (e.g., via electrode 590 by measuring impedance) or by advective flow of sweat (e.g., via sensor 524 by thermal mass flow sensing). The controller may only implement electroporation when, for example, the sweat generation rate is between about 0.1 to 0.5 nL/min/gland. Similarly, in an embodiment, the amount of electroporation could be increased or decreased as sweat generation rate increases or decreases, respectively.

While the above embodiments are described relative to sweat, embodiments of the disclosed invention are not so limited. Saliva is a biofluid that is similar to sweat and is diluted of larger molecules and proteins. Saliva does not necessarily require stimulation (i.e., it is always flowing), so a device for sensing saliva could use or not use a stimulation method, such as those described above as specified for sweat. With further reference to FIG. 4, a device could be placed in the mouth, with the device containing the sensing component 120. The skin 12 represents the tissue lining the mouth, which could be electroporated. In other words, when the biofluid to be sensed is saliva, references to “the skin” may include the oral mucosa or other tissue in the mouth. When such a device is placed underneath the tongue, salivary glands could be electroporated via the electrodes 290, 195 similar to that described for eccrine sweat glands. Larger analytes could then partition into the saliva and be sensed by the sensors 220, 222. With saliva, the wicking components 230, 232 may not be useful as saliva generation rates are generally much higher than sweat generation rates. In that regard, fresh saliva could be provided to sensors quickly and be displaced as new saliva appears without the function of a wicking component.

With reference to FIG. 8, in an embodiment, a saliva sensing device 600 is shown where like numerals and features in the device 100 are those also found in the device 600. The device 600 is positioned on the oral mucosa 18. The oral mucosa 18 is in a location where salivary glands exist (e.g., under the tongue). A substrate 617 carries a wicking component 632, sensors 620, 622, and electroporation electrode 690. The electroporation is achieved by the electroporation electrode 690 and a counter electrode 695. The electrode 690 and the sensors 620, 622 may be at the same or similar potential, as described previously for the sweat sensing example. The wicking component 632 could be, without limitation, a hydrophilic textile, sponge, polymer or other component that allows newly generated saliva to reach the sensors 620, 622. As noted previously for sweat sensing, additional mechanical housing, adhesives, or other features known to those skilled in the art could be added and arranged as needed.

Saliva monitoring devices could be mechanically less comfortable or ergonomic for longer term use than sweat monitoring devices. However, because saliva is always generating in the mouth, it could be suitable for one-time biomarker analysis. As a result, a collection-only device (similar to that described previously for sweat) may be useful.

With reference to FIG. 9, in an embodiment, a device 700 is depicted where like numerals and features correspond to those shown in previous figures. The device 700 includes a substrate 717 that carries a wicking component 730 and electrode 790. The device 700 includes an adhesive material 750 that adheres the device 700 to the oral mucosa 18. The wicking component 730 is configured to collect and hold a volume of saliva that could range from 10's of nL to the mL sample range. The substrate 717 could be a saliva impermeable polymer, such as PET, which prevents old saliva from mixing with newly generated saliva underneath the collection device 700. The oral adhesive material 750 helps decrease or eliminate the mixing of old and new saliva in the mouth. The oral adhesive material 750 could be any appropriate oral adhesive as known by those skilled in the art that allows ease of placement, securing, and removal of the device 700 as needed. In an embodiment, the electroporation achieved by the electrodes 790, 795 occurs before or during the collection of saliva by the device 700. The counter electrode 795 could also be exposed to the oral cavity, where through saliva it would provide a large area of electrical contact to the tissue of the mouth and, therefore, a lower and less noticeable current density through the tissue.

Embodiments of the disclosed invention apply at least to any type of biofluid sensor device that stimulates and measures biofluid (e.g., sweat, saliva, and tears), solutes within the biofluid, solutes that transfer into the biofluid from skin, a property of or things on the surface of skin, or properties or things beneath the skin. The disclosed invention applies to sweat sensing devices that can take on forms including patches, bands, straps, portions of clothing, wearables, or any suitable mechanism that reliably brings sweat stimulating, sweat collecting, and/or sweat sensing technology into intimate proximity with sweat as it is generated. Some embodiments of the disclosed invention utilize adhesives to hold the device near the skin, but devices could also be held by other mechanisms that hold the device secure against the skin, such as a strap or embedding in a helmet. Certain embodiments of the disclosed invention show sensors as simple individual elements. It is understood that many sensors require two or more electrodes, reference electrodes, or additional supporting technology or features which are not explicitly described in the description herein. Sensors are preferably electrical in nature, but may also include optical, chemical, mechanical, or other known biosensing mechanisms. Sensors can be in duplicate, triplicate, or more, to provide improved data and readings. The above description of various embodiments of the disclosed invention may not include a description of each and every component that may be required for the functioning of the devices depending on the application (e.g., a battery, or a counter electrode for iontophoresis), although it should be recognized that such components are included in the scope of the disclosed invention. For the purpose of brevity and to provide a focus on the inventive aspects described above, such components are not explicitly shown in the diagrams or included in the relevant description.

The following examples are provided to help illustrate the disclosed invention, and are not comprehensive or limiting in any manner.

### Example 1

Vasopressin, also known as antidiuretic hormone, is a neurohypophysial hormone related to hydration. Vasopressin is an analyte that is roughly 1,000 Da in molecular weight and, therefore, can become diluted in sweat compared to smaller lipophilic molecules such as cortisol. A device according to an embodiment of the disclosed invention could constantly apply a mild level of electroporation to increase the concentration of vasopressin extracted in sweat. Consider first the use of periodic electroporation for monitoring dehydration. In such an application, it may be desirable to track water loss through measuring sweat generation rate at an unstimulated sweat sensing site (e.g., by measuring sodium (Na) concentration, skin impedance, and/or using a flow meter) and also through measuring a dehydration biomarker (e.g., vasopressin) every hour. In a case where skin impedance, Na+, urea, and vasopressin are being measured to monitor dehydration, vasopressin might be the only measured analyte where the concentration in sweat would significantly increase due to electroporation. Because the measurements are hourly, there would be no need to continuously electroporate. Therefore, the electroporation—which may be preceded by sweat stimulation if needed—could be applied for 10 minutes each hour, which would be only ⅙ of the total electroporation time compared to continuous electroporation. As a result, the total amount of electroporation is dramatically reduced.

### Example 2

Luteinizing hormone, also known as lutropin and sometimes lutrophin, is a hormone produced by gonadotropic cells in the anterior pituitary gland, and in women is a marker of ovulation. Luteinizing hormone is large at around 30,000 Da. A device according to an embodiment of the disclosed invention could apply a moderate level of electroporation for a short period of time once a day to enhance the extraction of the luteinizing hormone through sweat and, therefore, the sensing of the luteinizing hormone in the generated sweat.

Consider next the use of on-demand electroporation for measuring the luteinizing hormone for fertility monitoring. A new device or a new disposable portion of a device could be applied each day. The device may measure estrogen and progesterone in sweat or some other biomarker, e.g. Cl− concentration, which can be used to indicate the body's thermal set-point and, in turn, impending ovulation. The device could on demand implement electroporation to increase the concentration of luteinizing hormone in sweat. On demand electroporation could be initiated at a set time each day or at an opportune time. For example, in an embodiment, the electroporation could be initiated by the user at an opportune time. As a result, in some cases, electroporation for a user may only occur once or very few times per month. Electroporation could also be implemented automatically based on feedback such as measurements of progesterone, for example.

### Example 3

Consider an embodiment where electroporation is applied to enable a sweat sensing device to measure a large protein that has a negative zeta potential at the pH and salinity of sweat. In an embodiment, a 10 ms DC electroporation pulse of +0.2 to +0.4 V could be applied once every second to the electroporation electrode ( 1/100 duty cycle) to help transport this protein into the sweat duct. The pulse may be applied once a second because, for example, roughly one second is required before this negatively charged protein is able to repopulate its concentration near the permeabilized locations of the eccrine duct. In an embodiment where both negatively and positively charged proteins are targeted for electroporation, the above example pulse could be bipolar in nature (e.g., +/−0.2 to +/−0.4 V, 1/50 duty cycle).

### Example 4

This example shows how different pulse magnitudes and widths can be used to control the depth of electroporation into the sweat gland. For example, it may be preferable to only or primarily electroporate the dermal duct so as to not interfere with the secretory portion of the gland where sweat is generated. A basic electrical model may be used to determine the desired pulse duration to get the applied voltage to the skin to be 80% of its applied level along the entire length of the dermal duct. An RC time constant for the dermal duct can be estimated as 33 Mohm*0.03 nF=0.0009 s, or about 1 ms (for 63.2% change in voltage). The cutoff frequency is therefore 1/(2*Pi*1 ms)=160 Hz. The rise time of the pulse to ensure that 80% of voltage gets to the bottom of the dermal duct is therefore 1.4 ms using the same RC time-constant calculation based on an inverse exponential trend.

This basic electrical model can be further explored. The dermis is mainly open space (collagen) filled with interstitial fluid. If one assumes an average of two layers of cells in the dermal duct lining, there are four lipid bilayers to electroporate. Assuming the full capacitance between the sweat duct and a counter electrode, the actual capacitance due to the presence of many lipid bilayers could be about 4× smaller (e.g., requiring a pulse of about 350 μs, per the calculations above). Also, if much of the field is vertical (i.e., downward into the skin), the capacitance will increase deeper inside the duct. A conventional target for electroporation of stand-alone cells is often 10 μs, but in the context of electroporating sweat glands, the required pulse may be longer in some instances to achieve deeper penetration of electroporation into the sweat gland.

If the voltage is increased, the pulse width decreases non-linearly, so that higher voltages could require less total wattage, reducing electrical stress on the skin. For example, starting with the 350 μs pulse calculated above, the time to charge the bottom of the dermal duct to 10% of the applied voltage is 35 μs, using V=Vapp*(1−e^{(-tRC)}. Therefore, if shorter pulses are needed for the electroporation waveform, the applied voltage may be raised by 10×. If the initial electroporation target voltage was about 2 V (for four bilayers), then the increased voltage would be about 20 V to charge the bottom of the duct to 2 V, which is the maximum voltage used for conventional iontophoresis (typical range is 12 to 15 V). Now, assume 8 bipolar pulses of 35 μs each. That would be less than 1 ms of total electrical stress, which is 120,000× less electrical stress than the 2 minutes used for Nanoduct sweat stimulation.

In another example, it may be desirable to only electroporate the first third of the dermal duct (e.g., 0.66 mm deep from the skin surface). Assume a sweat rate of 0.3 nL/min gland, which requires 1 minute for the sweat to traverse this upper third of the dermal duct, and assume the sweat is always within 7.5 μm distance from the duct wall (i.e., very close). In this case, the capacitance goes down by 3×, the resistance goes down by 3×, and the RC time constant is almost 10× lower. Therefore, in this example, the electroporation pulse can be reduced to only 3.5 μs.

As a further example, a plurality of different electroporation pulses could be applied, each pulse having a higher or lower voltage magnitude and a higher or longer pulse duration than the previous pulse. For example, a DC ramp could first be applied for about 200 μs, which enables more of the sweat gland depth to be at equipotential and near the threshold for electroporation compared to the natural state. Then, a higher frequency voltage could be applied with shorter pulsing (e.g., 10's of μs) to cause the electroporation. After this, one or more chaser waveforms could then be applied. In this example, the electroporation pulse would be superimposed on a lower frequency or DC waveform.

While specific embodiments have been described in considerable detail to illustrate the present invention, the description is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope of the general inventive concept.

What is claimed is:

1. A method of collecting and sensing a biofluid with enhanced concentration of analytes due to electroporation, comprising:
electroporating biofluid glands that are generating a biofluid; and
specifically sensing at least one analyte in said biofluid, the at least one analyte having a molecular weight greater than 50 Da.

2. The method of claim 1, wherein the biofluid glands are sweat glands, the method further comprising:
stimulating sweat prior to electroporating the sweat glands.

3. The method of claim 1, wherein electroporating includes first applying an electroporation waveform followed by applying a chaser waveform.

4. The method of claim 1, further comprising:
determining an amount of electroporation incurred by the biofluid glands.

5. The method of claim 4, wherein determining includes measuring at least one of a skin impedance, a change in an analyte concentration, or an advective flow of biofluid from the biofluid glands using a sensor.

6. The method of claim 5, wherein the sensor communicates with at least one electroporation element, the method further comprising:
controlling the electroporation via the at least one electroporation element based on feedback from the sensor.

7. The method of claim 5, wherein determining comprises:
measuring a first skin impedance prior to electroporating; and
measuring a second skin impedance during or after electroporating,
wherein electroporating causes the second skin impedance to decrease by no more than at least one of 20%, 50%, or 80% of the first skin impedance.

8. The method of claim 1, wherein electroporating the biofluid glands includes applying electroporation pulses at a plurality of unique locations on the skin.

9. The method of claim 1, wherein electroporating includes applying electroporation pulses having a duration that is less than at least one of the following: 10 s; 1 s; 100 ms; 10 ms; 1 ms; 100 μs; 10 μs; or 1 μs.

10. The method of claim 1, further comprising:
determining whether an electroporation electrode is in electrical contact with the biofluid glands prior to electroporating the biofluid glands.

11. The method of claim 1, wherein electroporating the biofluid glands causes an increase in concentration of said at least one analyte in a biofluid sample, wherein at least ⅔ of said concentration increase originates from electroporation of the biofluid glands in skin.

12. The method of claim 1, wherein electroporating the biofluid glands causes an increase in concentration of said at least one analyte in a biofluid sample, wherein at least 9/10 of said concentration increase originates from electroporation of the biofluid glands in skin.

13. The method of claim 1, wherein electroporating includes applying electroporation pulses periodically.

14. The method of claim 1, wherein electroporating includes applying electroporation pulses on-demand.

15. The method of claim 1, wherein electroporating includes applying electroporation pulses that include both negative and positive polarities.

16. The method of claim 1, wherein electroporating includes applying at least one electroporation waveform.

17. The method of claim 16, wherein electroporating includes applying at least one chaser waveform before, during, or after applying the at least one electroporation waveform.

18. The method of claim 1, wherein sensing includes sensing a first analyte in the biofluid and sensing a second analyte in the biofluid different from the first analyte the method further comprising:
comparing a concentration of the first analyte with a concentration of the second analyte.

19. A method of collecting and sensing saliva with enhanced concentration of analytes due to electroporation, comprising:
electroporating saliva glands; and
specifically sensing at least one analyte in said saliva.

20. The method of claim 19, wherein electroporating includes first applying an electroporation waveform followed by applying a chaser waveform.

21. The method of claim 19, further comprising:
measuring the amount of electroporation using a sensor that communicates with at least one electroporation component.

22. A device wearable on a user's skin for receiving an advective flow of a biofluid, wherein the biofluid is one of sweat, saliva, and tears, comprising:
at least one of a biofluid stimulation component, a biofluid sensor specific to an analyte, or a biofluid collection element;
at least one electroporation electrode for enhancing concentration of at least one analyte in the biofluid having a molecular weight of greater than 50 Da;
a counter electrode; and
an electroporation waveform generator configured to cause the electroporation electrode to generate and direct a plurality of electroporation pulses into the skin.

23. The device of claim 22, wherein each of the electroporation pulses are of a duration that is less than at least one of the following: 10 s; 1 s; 100 ms; 10 ms; 1 ms; 100 μs; 10 μs; or 1 μs.

24. The device of claim 22, further comprising:
at least one sensor for measuring an advective flow of biofluid from biofluid ducts in the skin.

25. The device of claim 24, wherein said at least one sensor for measuring an advective flow is in electrical communication with said at least one electroporation electrode.

26. The device of claim 22, wherein said electroporation electrode causes an increase in concentration of said at least one analyte in a biofluid sample, wherein at least ⅔ of said concentration increase originates from electroporation of biofluid ducts and biofluid glands in skin.

27. The device of claim 22, wherein said electroporation electrode causes an increase in concentration of said at least one analyte in a biofluid sample, wherein at least 9/10 of said concentration increase originates from electroporation of biofluid ducts and biofluid glands in skin.

28. The device of claim 22, further comprising:
at least one of the following components for reducing biofluid volume within the device: a wicking component or a biofluid-impermeable filling material.

29. The device of claim 28, wherein said wicking component is in fluid communication with said at least one electroporation electrode.

30. The device of claim 22, wherein said electroporation electrode is referenced to the same voltage potential as said at least one biofluid sensor.

31. The device of claim 22, wherein said electroporation electrode has a contact area with the skin that is less than a contact area with the skin of said counter electrode.

32. The device of claim 22, wherein said electroporation electrode has a contact area with the skin that is less than 0.05 cm², 0.1 cm², or 0.5 cm².

33. The device of claim 22, wherein said electroporation electrode is at least parallel to or partially surrounded by said counter electrode.

34. The device of claim 22, further comprising:
a plurality of electroporation electrodes and counter electrodes that are interspersed to localize an electric field.

35. The device of claim 22, further comprising:
at least one counter electrode that faces a first surface of the skin,
wherein said electroporation electrode faces a second surface of the skin that is rotated 90 degrees or more away from the first surface of the skin.

36. The device of claim 22, further comprising:
at least one of a numbing agent, an anti-inflammatory agent, or a pain-relieving agent.

37. The device of claim 22, further comprising:
at least one electroporation sensor for measuring an amount of electroporation incurred by biofluid ducts or biofluid glands in the skin.

38. The device of claim 37, wherein said electroporation sensor measures at least one of a skin impedance or a change in an analyte concentration.

39. The device of claim 37, wherein there is a first skin impedance without electroporation and a second skin impedance with electroporation, and said electroporation sensor and said electroporation electrode are in communication such that the second skin impedance is decreased by no more than at least one of 20%, 50%, or 80% of the first skin impedance.

40. The device of claim 22, wherein there are at least two analyte-specific sensors for measuring the concentration of at least two analytes in a biofluid sample, where said analytes are increased in concentration in the biofluid sample by electroporation.

41. The device of claim 22, wherein said at least one electroporation electrode generates at least one of: an electroporation waveform or a chaser waveform.

42. The device of claim 22, where said electroporation electrode is in communication with at least one sensor for: an analyte concentration, a skin impedance, a biofluid pH, or a biofluid salinity.
